(19) United States
(12) Patent Application Publication
(10) Pub. No.: US 2005/0143600 A1
(43) Pub. Date: Jun. 30, 2005

(54) Process for the preparation of 2-hydroxy carboxylic acids

(76) Inventors: Raghunath Vitthal Chaudhari, Pune (IN); Sunil Sopana Tonde, Pune (IN)

Correspondence Address: BAKER & BOTTS, 30 ROCKEFELLER PLAZA, NEW YORK, NY, 10112, US

(21) Appl. No.: 10/808,965

(22) Filed: Mar. 25, 2004

Related U.S. Application Data
(63) Continuation of application No. PCT/IB03/06202, filed on Dec. 26, 2003.

(51) Int. Cl. C07C 51/12

(52) U.S. Cl. 562/519

(57) ABSTRACT

A two step preparation for hydroxy carboxylic acid (e.g. lactic acid) is disclosed. An enol ester (e.g. vinyl acetate) is carbonylated in the presence of a hydroxyl compound (e.g. methanol) using a palladium catalyst having one or more O-, N- and/or P-containing ligands (e.g. PdCl₂(PPh₃)₂), and a solvent at 50-150° C./50-2000 psig to yield hydroxy ester (e.g. methyl lactate) and acetoxy ester (e.g. methyl-2-acetoxy propionate). The second step involves hydrolyzing the products of the carbonylation step using acid catalysts (e.g. TsOH, aq HCl, resin) at 10-125° C. to produce 2-hydroxy carboxylic acids (e.g. lactic acid). The carbonylation and hydrolysis catalysts may be separated and recycled.

### CROSS-REFERENCE TO RELATED APPLICATIONS

This is a continuation of International Application No. PCT/IB2003/006202, filed Dec. 26, 2003, which is incorporated by reference herein in its entirety.

### FIELD OF THE INVENTION

The present invention relates to a process for the preparation of 2-hydroxy carboxylic acids. Particularly the present invention relates to a process wherein an enol ester and a hydroxyl compound react with carbon monoxide in presence of a palladium catalyst, containing one or more ligands having one or more coordinating N, O and or P atoms and a solvent at a temperature and a pressure, to produce the 2-acetoxy ester and/or 2-hydroxy ester of the corresponding carboxylic acid, which on further catalytic hydrolysis results in the 2-hydroxy carboxylic acid. The process has potential importance when applied to vinyl acetate. In a preferred embodiment, vinyl acetate reacts with a hydroxyl compound and carbon monoxide to yield a 2-acetoxy propionic acid or 2-acetoxy propionate ester and/or lactate ester, which can be converted to lactic acid on hydrolysis. Lactic acid is important commercially in various industries, such as baking, cheese, wool dying, resin plasticiser, etc.

### BACKGROUND OF THE INVENTION

Lactic acid has been produced industrially by fermentation of molasses. However, the process is costly and inefficient due to the large amount of byproducts generated by the process. It is the product separation and purification that is expensive. Another commercial source of lactic acid is hydrocyanation of acetaldehyde, followed by hydrolysis of cyanohydrin with H₂SO₄. This method is highly corrosive, consuming stoichometric amount of toxic HCN and H₂SO₄. Furthermore, the process uses expensive HCN and produces stoichometric amount of (NH₄)₂SO₄.

Alkoxycarbonylation of certain aceloxyolefinic compounds is reported in U.S. Pat. Nos. 4,257,973 and 3,857,319.

German patent 1,221,224 and Swiss patent 589,571 disclose carbonylation of alcohols or phenols with CO and olefins. However, neither patent discloses the alkoxycarbonylation of enol acylates with CO and hydroxyl compound.

U.S. Pat. No. 4,072,709 provides a process for the production of lactic acid, in which alpha-aceloxy-propanaldehyde formed by hydroformylation of vinyl acetate is oxidized to alpha-aceloxy-propionic acid, which is further hydrolyzed to lactic acid. However, the process involves three steps for the formation of lactic acid.

U.S. Pat. No. 4,377,708 provides a process for hydrocarbonylation of vinyl acetate using CO and water as reactants with vinyl acetate. In the process, special precautions are taken to ensure the stability of the catalyst, reactants and products. The process requires the maintenance of a concentration of water at not more than 3 weight percent of the medium to avoid the hydrolysis of reactant vinyl acetate to acetic acid and acetaldehyde.

European patent 0144188 provides a process for alkoxycarbonylation of enol esters with hydroxyl compounds using Pd, Rh and Ni catalysts and further hydrolysis of the products to hydroxy acids. However, the process operates at a low concentration of hydroxyl compound (<10 times of enol ester). In addition, the process does not provide catalyst separation method and reuse, showing inefficiency of the catalyst.

Palladium catalyzed hydrocarbonylation of enol esters has been reported in Bull Chem. Soc. Jpn. 69, 1337-1345 (1996). However, the disclosed process requires high pressure of CO (150-250 atm.) and a base, such as pyridine or its derivatives. In addition, loading of the catalyst is high (5 mol % of enol ester) which gives less activity in terms of turn overnumber. Also the process is applicable only for acetoxy esters and hydroxy esters, and not for the important product like hydroxy acids such as lactic acid.

As can be seen, the prior art processes suffer from several disadvantages such as use of costly and toxic chemicals, formation of large amount of byproducts, low catalyst activity, and catalyst and reactant stability. It is therefore necessary to develop a process for preparation of 2-hydroxy carboxylic acids, which overcomes the drawbacks enumerated above.

The main object of the present invention is to provide a process for the preparation of 2-hydroxy carboxylic acids, which overcomes the drawbacks of low activity, catalyst stability, use of toxic chemicals, and severe operating conditions.

Another object of the invention is to provide an efficient catalytic process for the preparation of 2-hydroxy carboxylic acids via carbonylation of enol ester and subsequent hydrolysis of ester of the corresponding 2-acetoxy carboxylic acid and/or ester of the corresponding 2-hydroxy carboxylic acid that operates at milder reaction condition.

Still another object of the present invention is to provide the methods for catalyst separation and reuse.

### SUMMARY OF THE INVENTION

The present invention relates to a process for preparing a hydroxy carboxylic acid comprising using a palladium catalyst, having one- or more ligands containing coordinating nitrogen and/or oxygen and/or phosphorus atoms, to catalyze carbonylation of an enol ester in the presence of a hydroxyl compound to yield 2-acetoxy ester and/or 2-hydroxy ester of the corresponding carboxylic acid at milder reaction conditions, which on further hydrolysis, using acid catalysts, gives a 2-hydroxy carboxylic acid. Both carbonylation and hydrolysis catalysts are reusable.

### DETAILED DESCRIPTION OF THE PRESENT INVENTION

Accordingly the present invention provides a process for the preparation of 2-hydroxy carboxylic acids, comprising
a) Carbonylating an enol ester with carbon monoxide and a hydroxyl compound in the presence of a palladium catalyst comprising an oxygen and/or nitrogen and/or phosphorus containing ligand(s) and a solvent, at a temperature in the range of 50-250° C., at a pressure in the range of 50-2000 psig, to obtain a carbonylated ester;
b) hydrolyzing the carbonylated ester with an acid catalyst at a temperature of 10-125° C., to obtain the 2-hydroxy carboxylic acid.

In an embodiment of the invention, the molar concentration ratio of enol ester to catalyst is in the range of 25:1 to 1,000:1.

In another embodiment of the invention, the molar concentration ratio of hydroxyl compound and enol ester is not less than one.

In another embodiment of the invention, the carbonylation catalyst may be recycled for the carbonylation step.

The enol ester may be an organic compound having formula R₁C═C(R₂)—O-Acyl, where R₁ is H or an alkyl group containing 1-5 carbon atoms and R₂ is H or an alkyl group containing 1-5 carbon atoms.

The hydroxyl compound may be a compound having formula R—OH, where R is H or primary, secondary or tertiary alkyl group containing 1-7 carbon atoms. The hydroxyl compound is preferably selected from the group consisting of water, methanol, ethanol, propanol, iso-propanol, butanol, isobutanol, t-butanol, and pentanol.

The catalyst may comprise a palladium (II) or palladium (0) compound having formula ABxCy, where A stands for palladium, B is an organic ligand containing one or more coordinating nitrogen and/or oxygen and/or phosphorus atoms and C is any halogen atom such as F, Cl, Br or I and (x+y) is an integer ranging from 1 to 4, wherein individually x and y can vary in the range of 0 to 4. Such palladium compounds can be selected from the group consisting of palladium chloride, palladium bromide, palladium iodide, and palladium acetate; or a metal complex of palladium such as bis(acetylacetonato)palladium (II), bis(triphenylphosphine)dichloropalladium (II), bis(triphenylphosphine)dibromopalladium (II), bis(triphenylphosphine)diiodopalladium (II), bis(pyridine)dichloropalladium(II), bis(pyridine)didromopalladium (II), bis(pyridine)diiodopalladium (II), bis(acetonotrile)dichloropalladium (II), bis(benzonitrile)dichloropalladium (II), and tetrabis(triphenylphosphine)palladium (0).

The organic ligand is a compound containing one or more coordinating O atoms selected from the group consisting of acetyl acetonate, salicylaldehyde, p-toluenesulphonic acid, compounds containing one or more coordinating N atoms, such as pyridine, pipyridine, triethyl amine, tributyl amine, quinoline, isoquinoline, O-phenylenediamine, p-phenylenediamine, ethylenediamine, or coordinating N and O atoms, such as 8-hydroxy quinoline, bis(saliylidene)ethylenediamine, salicylaldoxime, picolinic acid, nicotinic acid, anthranilic acid, one or more P containing compound such as trimethyl phosphine, triethyl phosphine, tri-n-butyl phosphine, tri-t-butyl phosphine, tricyclohexyl phosphine, triphenyl phosphine, bis(dicyclohexylphosphinoethane), bis(dicyclohexylphosphinobutane), bis(diphenylphosphinoethane), bis(diphenylphosphinopropane), bis(diphenylphosphinobutane), bis(diphenylphosphinohexane).

The solvent may be an organic solvent selected from toluene, benzene, chloroform, dichloromethane, dichloroethane, chlorobenzene, o-dichlorobenzene, p-dichlorobenzene a ketone, e.g. acetone, ethyl methyl ketone, diethyl ketone, acetophenone, a cyclic ether, e.g. tetrahydrofuran, dioxan, or a nitrile, e.g. acetonitrile or benzonitrile.

In another embodiment of the invention, the carbonylation product is separated by vacuum distillation or solvent extraction using an appropriate solvent, and the carbonylation catalyst is recycled and reused for the carbonylation step.

Hydrolysis of carbonylation products may be carried out with a catalyst, particularly an acidic catalyst, such as p-toluene sulphonic acid, and aqueous hydrochloric acid, or a resin, such as amberlite, at a temperature in the range of 10-125° C. The catalyst may be separated by distillation or filtration and reused for hydrolysis.

The process of the invention provides an alternative catalytic system for the production of lactic acid, which is both economic and efficient. The process also provides for catalyst separation and reuse. In addition, the process operates at milder reaction conditions than processes disclosed by the prior art.

### EXAMPLES

### Example 1

A 50 ml autoclave was charged with the following:

| | | |
|---|---|---|
| Vinyl acetate: | 0.025 | mol |
| Methanol: | 0.060 | mol |
| PdCl₂(PPh₃)₂: | 0.00005 | mol |
| Acetyl acetone: | 0.001 | mol |
| Toluene: | 20 | ml |

The contents of the autoclave were flushed thrice with carbon monoxide at room temperature. Thereafter, the contents were heated at 100° C. The autoclave was pressurized with carbon monoxide to 800 psig after the temperature was attained. The contents were stirred for 4 hours continuously. The reactor was then cooled to room temperature and the gas was vented off. The liquid contents were analyzed by gas chromatography. The results of the gas chromatography showed 83.5% conversion of vinyl acetate with 47.92% selectivity to methyl-2-acetoxy propionate and 8.0% selectivity to methyl lactate with turnover number of 210. (TON=Moles of the products hydrolyzable to lactic acid per mole of the catalyst charged).

The product methyl-2-acetoxy propionate was characterized by ¹H-NMR spectroscopy after separation by evaporating the low boilers and solvent and filtering out the precipitated catalyst.

### Example 2

A 50 ml autoclave was charged with the following:

| | | |
|---|---|---|
| Vinyl acetate: | 0.025 | mol |
| Methanol: | 0.060 | mol |
| PdCl₂(PPh₃)₂: | 0.00005 | mol |
| Picolinic acid: | 0.001 | mol |
| Toluene: | 20 | ml |

The contents of the autoclave were flushed thrice with carbon monoxide at room temperature. Thereafter, the contents were heated at 100° C. The autoclave was pressurized with carbon monoxide to 800 psig after the temperature was attained. The contents were stirred for 10 hours continuously. The reactor was then cooled to room temperature and the gas was vented off. The liquid contents were analyzed by gas chromatography. The results of the gas chromatography showed 97.66% conversion of vinyl acetate with 61.42% selectivity to methyl-2-acetoxy propionate and 18.98% selectivity to methyl lactate with turn over number of 399.4.

### Example 3

Catalyst for recycle run was obtained by filtration after evaporating the low boilers and solvent from the reaction mixture of example 2.

A 50 ml autoclave was charged with the following:

| Vinyl acetate: | 0.025 | mol |
|---|---|---|
| Methanol: | 0.060 | mol |
| Catalyst: | recycled from example 2 | |
| Picolinic acid: | 0.001 | mol |
| Toluene: | 20 | ml |

The contents of the autoclave were flushed thrice with carbon monoxide at room temperature. Thereafter, the contents were heated at 100° C. The autoclave was pressurized with carbon monoxide to 800 psig after the temperature was attained. The contents were stirred for 10 hours continuously. The reactor was then cooled to room temperature and the gas was vented off. The liquid contents were analyzed by gas chromatography. The results of the gas chromatography showed 63.53% conversion of vinyl acetate with 38.08% selectivity to methyl-2-acetoxy propionate and 15.67% selectivity to methyl lactate.

### Example 4

A 50 ml autoclave was charged with the following:

| | | |
|---|---|---|
| Vinyl acetate: | 0.025 | mol |
| Methanol: | 0.060 | mol |
| PdCl₂(PPh₃)₂: | 0.00005 | mmol |
| Nicotinic acid: | 0.001 | mol |
| Toluene: | 20 | ml |

The contents of the autoclave were flushed thrice with carbon monoxide at room temperature. Thereafter, the contents were heated at 100° C. The autoclave was pressurized with carbon monoxide to 800 psig after the temperature was attained. The contents were stirred for 4 hours continuously. The reactor was then cooled to room temperature and the gas was vented off. The liquid contents were analyzed by gas chromatography. The results of the gas chromatography showed 60.15% conversion of vinyl acetate with 57.57% selectivity to methyl-2-acetoxy propionate and 16% selectivity to methyl lactate with turn over number of 227.

### Example 5

A 50 ml autoclave was charged with the following:

| | | |
|---|---|---|
| Vinyl acetate: | 0.025 | mol |
| Methanol: | 0.060 | mol |
| PdCl₂(PPh₃)₂: | 0.00005 | mol |
| Anthranilic acid: | 0.001 | mol |
| Toluene: | 20 | ml |

The contents of the autoclave were flushed thrice with carbon monoxide at room temperature. Thereafter, the contents were heated at 100° C. The autoclave was pressurized with carbon monoxide to 800 psig after the temperature was attained. The contents were stirred for 10 hours continuously. The reactor was then cooled to room temperature and the gas was vented off. The liquid contents were analyzed by gas chromatography. The results of the gas chromatography showed 98.9% conversion of vinyl acetate with 50.30% selectivity to methyl-2-acetoxy propionate and 20% selectivity to methyl lactate with turn over number of 356.

### Example 6

A 50 ml autoclave was charged with the following:

| | | |
|---|---|---|
| Vinyl acetate: | 0.025 | mol |
| Methanol: | 0.060 | mol |
| PdCl₂(PPh₃)₂: | 0.00005 | mol |
| Pyridine: | 0.001 | mol |
| p-toluenesulphonic acid: | 0.0002 | mol |
| Toluene: | 20 | ml |

The contents of the autoclave were flushed thrice with carbon monoxide at room temperature. Thereafter, the contents were heated at 100° C. The autoclave was pressurized with carbon monoxide to 800 psig after the temperature was attained. The contents were stirred for 4 hours continuously. The reactor was then cooled to room temperature and the gas was vented off. The liquid contents were analyzed by gas chromatography. The results of the gas chromatography showed 99% conversion of vinyl acetate with 76.45% selectivity to methyl-2-acetoxy propionate with turn over number of 411.

### Example 7

A 50 ml autoclave was charged with the following:

| | | |
|---|---|---|
| Vinyl acetate: | 0.025 | mol |
| Methanol: | 0.060 | mol |
| PdCl₂(PPh₃)₂: | 0.00005 | mol |
| Triphenylphosphine: | 0.001 | mol |
| Toluene: | 20 | ml |

The contents of the autoclave were flushed thrice with carbon monoxide at room temperature. Thereafter, the contents were heated at 100° C. The autoclave was pressurized with carbon monoxide to 800 psig after the temperature was attained. The contents were stirred for 8 hours continuously. The reactor was then cooled to room temperature and the gas was vented off. The liquid contents were analyzed by gas chromatography. The results of the gas chromatography showed 99% conversion of vinyl acetate with 2% selectivity to methyl-2-acetoxy propionate with turn over number of 10.

### Example 8

A 50 ml autoclave was charged with the following:

| | | |
|---|---|---|
| Vinyl acetate: | 0.025 | mol |
| Methanol: | 0.060 | mol |
| PdCl₂(PPh₃)₂: | 0.00005 | mol |
| p-toluenesulphonic acid: | 0.0002 | mol |
| Acetyl acetone: | 0.001 | mol |
| Tetrahydrofuran | 20 | ml |

The contents of the autoclave were flushed thrice with carbon monoxide at room temperature. Thereafter, the contents were heated at 100° C. The autoclave was pressurized with carbon monoxide to 800 psig after the temperature was attained. The contents were stirred for 4 hours continuously. The reactor was then cooled to room temperature and the gas was vented off. The liquid contents were analyzed by gas chromatography. The results of the gas chromatography showed 91.43% conversion of vinyl acetate with 35.67% selectivity to methyl-2-acetoxy propionate and 25.65% selectivity to methyl lactate with turn over number of 295.

### Example 9

A 50 ml autoclave was charged with the following:

| | | |
|---|---|---|
| Vinyl acetate: | 0.025 | mol |
| Methanol: | 0.060 | mol |
| PdCl₂(PPh₃)₂: | 0.00005 | mol |
| Acetyl acetone: | 0.001 | mol |
| p-toluenesulphonic acid: | 0.0002 | mol |
| Tetrahydrofuran | 20 | ml |

The contents of the autoclave were flushed thrice with carbon monoxide at room temperature. Thereafter, the contents were heated at 100° C. The autoclave was pressurized with carbon monoxide to 800 psig after the temperature was attained. The contents were stirred for 4 hours continuously. The reactor was then cooled to room temperature and the gas was vented off. The liquid contents were analyzed by gas chromatography. The results of the gas chromatography showed 82.65% conversion of vinyl acetate with 4% selectivity to methyl-2-acetoxy propionate and 42.56% selectivity to methyl lactate with turn over number of 202.

### Example 10

A 50 ml autoclave was charged with the following:

| | | |
|---|---|---|
| Vinyl acetate: | 0.025 | mol |
| Methanol: | 23 | ml |
| PdCl₂(PPh₃)₂: | 0.00005 | mol |
| Acetyl acetone: | 0.001 | mol |

The contents of the autoclave were flushed thrice with carbon monoxide at room temperature. Thereafter, the contents were heated at 100° C. The autoclave was pressurized with carbon monoxide to 800 psig after the temperature was attained. The contents were stirred for 3 hours continuously. The reactor was then cooled to room temperature and the gas was vented off. The liquid contents were analyzed by gas chromatography. The results of the gas chromatography showed 91.26% % conversion of vinyl acetate with 3% selectivity to methyl-2-acetoxy propionate with turn over number of 10.97.

### Example 11

Methyl lactate was separated by extracting with 15 ml water from the reaction mixture of example 3, to which 1 ml of conc. HCl was added. Thereafter, the reaction mixture was refluxed for 3 hours. The contents were analyzed by gas chromatography after cooling the reaction mixture. The results showed 58% conversion of methyl lactate.

### Example 12

0.191 g of p-toluene sulphonic acid and 15 ml water was added to 1.46 g of methyl-2-acetoxy propionate. Thereafter, the reaction mixture was refluxed for 3 hours and the contents were analyzed by gas chromatography after cooling the reaction mixture. The analysis showed 100% conversion of methyl-2-acetoxy propionate with 100% selectivity to lactic acid.

### Example 13

15 ml of water and 0.1 g of amberlite IR 20 resin were added to the reaction mixture of example 4. Thereafter, the contents were heated to 80° C. for 3 hours. The contents were analyzed by gas chromatography. The analysis showed 17.33% conversion of methyl-2-acetoxy propionate.

### Example 14

The catalyst from example 13 was separated by filtration and added to 1.44 g of methyl-2-acetoxy propionate and 15 ml water. Thereafter the contents were heated to 80° C. for 3 hours. The analysis was done by gas chromatography. The results showed 41.77% conversion of methyl-2-acetoxy propionate with 100% selectivity to lactic acid.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. Various references are cited herein, the disclosure of which are incorporated by reference in their entireties.

1. A process for preparing a 2-hydroxy carboxylic acid using a reusable catalyst, said process comprises:
(a) carbonylating an enol ester with carbon monoxide and a hydroxyl compound in presence of a palladium catalyst and a solvent, at a temperature in the range between 50-250° C., at a pressure in the range of 50-2000 psig, to obtain a carbonylated ester, wherein the palladium catalyst comprises one or more organic ligands that contain atom selected from the group consisting of oxygen, nitrogen, and phosphorus and
(b) hydrolyzing the carbonylated ester with an acid catalyst at a temperature of 10-125° C. to obtain a 2-hydroxy carboxylic acid.

2. The process of claim 1, wherein the enol ester and the palladium catalyst are present in a molar concentration ratio in the range of 25:1 to 1,000:1.

3. The process of claim 1, wherein the hydroxyl compound and the enol ester are present in a molar concentration ratio not less than one.

4. The process of claim 1, wherein the carbonylation catalyst is recycled for the carbonylation step.

5. The process of claim 1, wherein the enol ester is an organic compound having formula R₁C═C(R₂)—O-Acyl, where R₁ is H or an alkyl group containing 1-5 carbon atoms and R₂ is H or an alkyl group containing 1-5 carbon atoms.

6. The process of claim 1, wherein the hydroxyl compound has a formula R—OH, wherein R is selected from the group consisting of H, a primary alkyl group containing 1-7 carbon atoms, a secondary alkyl group containing 1-7 carbon atoms, and a tertiary alkyl group containing 1-7 carbon atoms.

7. The process of claim 6, wherein the hydroxyl compound is selected from the group of consisting of water, methanol, ethanol, propanol, iso-propanol, butanol, iso-butanol, t-butanol and pentanol.

8. The process of claim 1, wherein the palladium catalyst is selected from palladium (II) having formula ABxCy or palladium (0) compound having formula ABxCy, wherein
A is palladium,
B is an organic ligand containing one or more coordinating nitrogen and/or oxygen and/or phosphorus atoms,
C is a halogen atom selected from the group consisting of F, Cl, Br and I,
x+y is an integer ranging from 1 to 4, and
x and y can vary independently in the range of 0 to 4.

9. The process of claim 1, wherein the palladium catalyst is selected from the group consisting of palladium chloride, palladium bromide, palladium iodide, palladium acetate, and metal complex of palladium.

10. The process of claim 9, wherein the metal complex of palladium is selcted from the group consisting of bis(acetylacetonato)palladium (II), bis(triphenylphosphine)dichloropalladium(II), bis(triphenylphosphine)dibromopalladium (II), bis(triphenylphosphine)diiodopalladium (II), bis(pyridine)dichloropalladium (II), bis(pyridine)didromopalladium (II), bis(pyridine)diiodopalladium (II), bis(acetonotrile)dichloropalladium (II), bis(benzonitrile)dichloropalladium (II), and tetrakis(triphenylphosphine)palladium (0).

11. The process of claim 1, wherein the organic ligand is a compound containing one or more coordinating O atoms selected from the group consisting of acetyl acetonate, salicylaldehyde, and p-toluenesulphonic acid.

12. The process of claim 1, wherein the organic ligand is a compound containing one or more coordinating N atoms selected from the group consisting of pyridine, pipyridine, triethyl amine, tributyl amine, quinoline, isoquinoline, o-phenylenediamine, and p-phenylenediamine, ethylenediamine.

13. The process of claim 1, wherein the organic ligand is a compound containing one or more coordinating N and O atoms selected from the group consisting of 8-hydroxy quinoline, bis(saliylidene)ethylenediamine, salicylaldoxime, picolinic acid, nicotinic acid, and anthranilic acid.

14. The process of claim 1, wherein the organic ligand is a compound containing one or more P atoms selected from the group consisting of trimethyl phosphine, triethyl phosphine, tri-n-butyl phosphine, phosphine, triphenyl phosphine, bis(dicyclohexylphosphinobutane), bis(diphenylphosphinopropane), and bis(diphenylphosphinohexane).

15. The process of claim 1, wherein the solvent is an organic solvent selected from the group consisting of toluene, benzene, chloroform, dichloromethane, dichloroethane, chlorobenzene, o-dichlorobenzene, p-dichlorobenzene, ketone, cyclic ether, and nitrile.

16. The process of claim 15, wherein the ketone is selected from the group consisting of acetone, ethyl methyl ketone, diethyl ketone, and acetophenone.

17. The process of claim 15, wherein the cyclic ether is selected from the group consisting of tetrahydrofuran and dioxan.

18. The process of claim 15, wherein the nitrile is selected from the group consisting of acetonitrile and benzonitrile.

19. The process of claim 1, further comprising separating by vacuum distillation or solvent extraction the palladium catalyst.

20. The process of claim 1, wherein the acid catalyst is selected from the group consisting of p-toluene sulphonic acid, aqueous hydrochloric acid, and a resin.

21. The process of claim 1, wherein the hydrolysis catalyst is recycled for the hydrolysis step.

22. The process of claim 1, further comprising separating by vacuum distillation or solvent extraction the acid catalyst.

22. The process of claim 1, wherein the 2-hydroxy carboxylic acid is lactic acid.
